(19) United States
(12) Patent Application Publication
(10) Pub. No.: US 2021/0332316 A1
(43) Pub. Date: Oct. 28, 2021

(54) SINGLE USE BIOREACTOR SYSTEM STERILIZABLE BY IRRADIATION AND METHOD FOR THE QUALITY ASSURANCE OF A SINGLE USE BIOREACTOR SYSTEM

(71) Applicant: SARTORIUS STEDIM BIOTECH GMBH

(72) Inventors: Christian GRIMM, Heilbad Heiligenstadt (DE); Gao WEI, Kassel (DE); Marco LEUPOLD, Kassel (DE)

(21) Appl. No.: 16/319,136

(22) PCT Filed: Jul. 3, 2017
(86) PCT No.: PCT/EP2017/066519

§ 371 Date: Jan. 18, 2019

(30) Foreign Application Priority Data
Jul. 20, 2016 (DE) 10 2016 113 412.0

(51) Int. Cl. C12M 1/00; C12M 1/36

(52) U.S. Cl. CPC C12M 23/28; C12M 41/48

(57) ABSTRACT

A single use bioreactor system, which is sterilizable by irradiation and provided for use in a biopharmaceutical process, comprises an RFID tag and a memory rewritable in principle, in which data with respect to an integrity check of the single use bioreactor system are stored. A method for the quality assurance of such a single use bioreactor system comprises the following steps: providing the single use bioreactor system with an RFID tag and a memory rewritable in principle, in particular of the type FeRAM, which preferably is part of the RFID tag; carrying out an integrity check of at least one component of the single use bioreactor system before a sterilization of the single use bioreactor system by irradiation; and writing data with respect to the integrity check into the memory before the sterilization of the single use bioreactor system.

The invention relates to a single use bioreactor system sterilizable by irradiation, which is provided for use in a biopharmaceutical process. The invention furthermore relates to a method for the quality assurance of such a single use bioreactor system.

The trends of recent years in the biopharmaceutical industry increasingly point in the direction of the use of single use components (SU components). The same no longer are used now only in the field of product and process development, but also in the field of clinical trial manufacturing (CTM) for the approval procedure and even in the commercial good manufacturing practice (GMP) in the production of drugs.

In the commercial production, such as for example in the cultivation of animal cells in a bioreactor, two different approaches of the utilization of single use components substantially are employed. On the one hand, especially in new factories, locations or enterprises in particular in Asia, the operators rely on a full single-use approach in which the entire manufacturing chain is covered almost exclusively by single use components. On the other hand, when classical appliances made of stainless steel are present already, the same in general shall also be utilized due to the high purchase price. In this case, so-called hybrid installations comprising existing stainless steel components and newly added single use components are being used.

In particular in the field of the clinical trial manufacturing or even in the surroundings of the method of checking the current quality standards in the field of manufacture (cGMP or Current Good Manufacturing Practice) it is indispensable that the integrity of the reaction vessel is given and thus both a penetration of extraneous organisms and a leakage of the reaction medium from the reactor can be prevented. In the case of stainless steel reactors this is ensured for example by applying a test pressure and determining the pressure drop over time as a measurement parameter, after cleaning, installation and steam sterilization. This pressure test is recorded by the operator of the installation and documented in a suitable way. In single use bioreactors or other systems made of plastics this is effected in a similar way directly before use. Directly after installation of the container, the system is aseptically connected to a pressure testing device via a hose connection. Testing instruments are available by means of which both pressure testing and a cGMP-compliant documentation can be ensured, e.g. the “Sartocheck 4 plus Bag Tester” of Sartorius.

As in contrast to stainless steel systems the single use systems are no fixed installations, the manufacture and the transport of the containers constitute a serious source of possible leakages. To prevent this from happening, a check of the container integrity in part is made already during the production of single use containers. For this purpose, the manufacturer employs systems similar to those being employed by the future user before their use. It is an essential disadvantage of the previous method that the information on the integrity test is not stored on the container itself and therefore must be traced back tediously via the lot number.

In another context a gamma-sterilizable RFID system is known from U.S. Pat. No. 8,963,684 B2, by means of which single-use bioprocess components not manufactured by the original manufacturer can be recognized and thereupon a non-authorized operation can be prevented. For this purpose an FeRAM chip (short for “ferroelectric random access memory chip”) is used in order to store error-correctable information on an RFID tag that is attached to a single-use bioprocess component. The information generally is preserved in the memory chip even after the gamma sterilization of the RFID tag and the single-use bioprocess component and can possibly be corrected. There is also described a method for authenticating the single-use bioprocess component, which is meant to reduce the liability risk of the original manufacturer. When a component does not pass an authentication test, a warning is issued to the user or the bioprocess is stopped. In this way, poor-quality counterfeits shall be recognized and users in this case shall be stopped from asserting an unjustified complaint with the original manufacturer.

It is the object of the invention to especially improve the documentation of the integrity check of a single use bioreactor system, which is used in a biopharmaceutical process, and to make it available more easily.

This object is solved by a single use bioreactor system comprising the features of claim 1 and by a method for the quality assurance of a single use bioreactor system comprising the features of claim 13. Advantageous and expedient embodiments of the single use bioreactor system according to the invention and of the method according to the invention are indicated in the respectively associated sub-claims.

The single use bioreactor system according to the invention is provided for use in a biopharmaceutical process and sterilizable by irradiation. The single use bioreactor system comprises an RFID tag and a memory rewritable in principle, in which data with respect to an integrity check of the single use bioreactor system are stored.

A single use bioreactor system is not necessarily understood to be a complete, fully functional system. The term “single use bioreactor system” here is representative of one or more essential single use components that are jointly provided for a use in a biopharmaceutical process. In particular, the single use bioreactor system shall comprise a container (bag) for use in a bioreactor.

The term RFID tag is not to be understood in a limiting sense and shall include all types of transponder units that are operable and/or readable in a wireless or contactless way and all wireless types and standards of transmission, such as NFC (Near Field Communication) etc.

The invention is based on the finding that there are electronic memories that survive the process of a sterilization of the single use bioreactor system by irradiation largely unharmed. This opens up the possibility of a dynamic documentation remaining on the product. This means that after a first storage of data in the memory, the information can be changed and/or completed after the radiation sterilization. Thus, the history of the single use bioreactor system can be documented carefully and be made available more easily for testing purposes.

The invention thus increases the process safety, as accurate data on the state or the relevant properties of the component(s) of the single use bioreactor system are available immediately and on the spot. On the one hand, this leads to less complaints with the manufacturer of the single use bioreactor system and on the other hand to an improvement of the supply chain.

The memory in which the data are stored before and after a radiation sterilization of the single use bioreactor system preferably is part of the RFID tag so that no separate electronic component must be integrated into the single use bioreactor system.

A memory suitable for the intended purposes is an FeRAM memory. It was found that this type of memory is largely resistant to the radiation (doses) used in the sterilization of single use components.

Advantageously, product-relevant data also are stored in the memory, in particular a product identification code and/or a date of manufacture and/or a use-by date. These data can relate to one or more components of the single use bioreactor system.

In an advantageous embodiment of the memory of the single use bioreactor system according to the invention, said memory includes a write protection that after its activation prevents deleting and overwriting of the data stored in the memory.

Advantageously, the memory can also be divided into a free, writable area and a blocked area that no longer is writable. In the blocked area, manufacturer data and information on the integrity check of the single use bioreactor system can be stored, which shall not be changed, while into the free memory information and/or measurement data can be written, which later on are determined by the customer e.g during the proper use of the single use bioreactor system.

It was found that certain types of battery survive the process of a sterilization of the single use bioreactor system by irradiation largely unharmed. Based on this surprising finding the RFID tag in an advantageous embodiment is provided with an internal battery. This battery can support the documentation of the history (production, integrity check, packaging, sterilization, storage, shipment) of the single use bioreactor system.

For the documentation and future evaluation of the integrity check and/or the storage conditions sensors advantageously can be used for the detection of certain events and/or ambient conditions that are part of the single use bioreactor system according to the invention.

The invention also creates an apparatus assembly comprising an accumulator unit attached to a packaging outside the single use bioreactor system, which in a releasable way is electroconductively connected to the RFID tag.

The accumulator unit provides for documenting the history of the single use bioreactor system over an extended period, in particular in the case of a long storage of the single use bioreactor system. As the accumulator unit is rechargeable, it can also serve for the power supply of the RFID tag and possible sensors of the single use bioreactor system after a possible discharge due to the radiation sterilization of the single use bioreactor system.

What is preferred particularly is an accumulator unit with a device for wireless charging by an external energy source. In this case, the accumulator unit can be charged in the packed condition of the single use bioreactor system, without having to create a cable connection or the like.

Preferably, the accumulator unit is attached to an inner side of an outer packaging, e.g. in an inside pocket of a carton. The interruption of the releasable electrical connection of the accumulator unit can be recognized and be documented as the time of unpacking and using the single use bioreactor system.

The apparatus assembly according to the invention can be completed by a writing device that is configured for the wireless writing of data into the memory of the single use bioreactor system, and a reading device that is configured for the wireless reading of data stored in the memory. Of course, the writing device and the reading device can be configured as a combined writing/reading device. Reading out the data and including the same into the process control also provides for a control function before the use of the single use bioreactor system. With reference to the data it can be ensured that the components used actually fit together and are compatible with each other. For example, after reading out the data a warning can be issued not to insert a 200-liter bag into a hardware that is designed for a 50-liter bag only.

The object of the invention also is solved by a method for the quality assurance of a single use bioreactor system that is provided for use in a biopharmaceutical process. The quality assurance method according to the invention comprises the following steps:
providing the single use bioreactor system with an RFID tag and a memory rewritable in principle, in particular of the type FeRAM, which preferably is part of the RFID tag;
carrying out an integrity check of at least one component of the single use bioreactor system before a sterilization of the single use bioreactor system by irradiation; and
writing data with respect to the integrity check into the memory before the sterilization of the single use bioreactor system.

With respect to the advantages of the method according to the invention reference is made to the corresponding foregoing explanations concerning the single use bioreactor system of the invention or the apparatus assembly of the invention.

As explained already, information on the performance of the integrity check and/or on storage conditions of the single use bioreactor system can be written into the memory in connection with a documentation of the history of the single use bioreactor system.

When the memory of the single use bioreactor system is equipped with a corresponding functionality, a write protection of the memory can be activated before shipment of the single use bioreactor system to a customer. Specific manufacturer data thereby remain protected.

According to a development of the idea underlying the invention the single use bioreactor system can record events and/or ambient conditions determined by means of one or more sensors and write associated data into the memory. The single use bioreactor system is supplied with energy via an external accumulator unit as long as the same can provide enough electric power.

Possibly, an internal battery of the RFID tag can take over the energy supply as soon as the energy of the accumulator unit falls below a certain limit value.

As explained already, the accumulator unit shall be configured such that after the radiation sterilization it can be charged again from outside in a contactless way by an external energy source.

According to a further continuation of the idea underlying the invention the electric line between the accumulator unit and the single use bioreactor system shall be interrupted and the time of this event shall be written into the memory upon opening of a packaging to which the accumulator unit is attached, upon removal of the single use bioreactor system from the packaging, or in the case of a defined concussion. This point in time shall document the unpacking and the use or the fact that from this point in time the integrity of the single use bioreactor system no longer is ensured on the part of the manufacturer.

Further features and advantages of the invention can be taken from the following description and from the attached drawing to which reference is made. In the drawings:

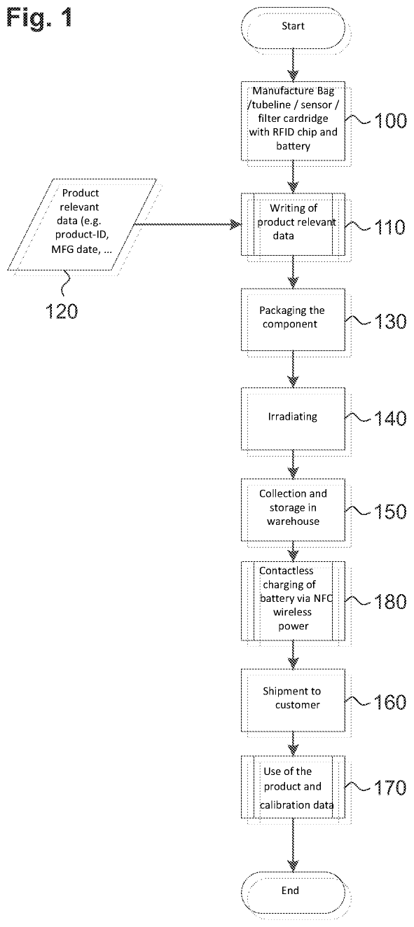

FIG. 1 by way of example shows the life cycle of a single use bioreactor system according to the invention;

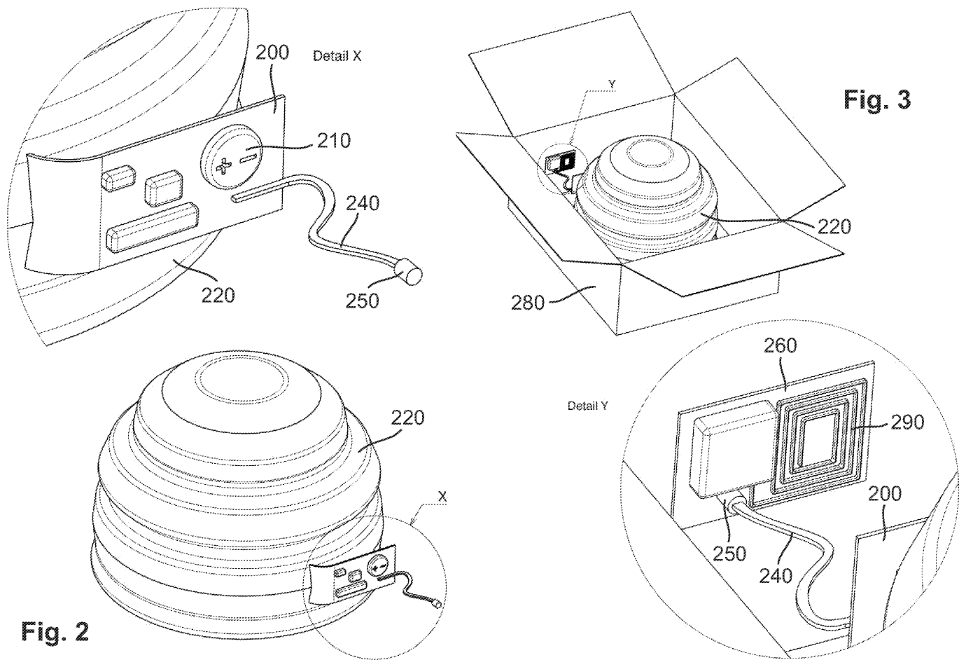

FIG. 2 shows a component of a single use bioreactor system with an RFID tag, including a detail view; and

FIG. 3 shows the component of FIG. 2 in a carton with a device for the wireless charging of an accumulator, including a detail view.

In the following, a typical life cycle of a single use bioreactor system is described with reference to the flow diagram shown in FIG. 1, starting with the step 100 of the manufacture of the single use bioreactor system. As components, the single use bioreactor system comprises for example a container (bag) for use in a bioreactor and a single use probe. At least the container is provided with an RFID tag (transponder), which in turn is provided with a memory rewritable in principle, in particular of the type FeRAM.

In a next step 110, product-relevant data 120 are wirelessly written into the memory of the RFID tag by using a suitable writing device. The product-relevant data can comprise a product identification code, the date of manufacture and further details related to the single use bioreactor system.

The components of the single use bioreactor system with the container are packed in step 130 and in step 140 sterilized by irradiation, in particular by gamma irradiation, as a complete unit, i.e. including the packaging. An FeRAM memory in generally survives a sterilization by irradiation, i.e. the data stored in the memory can still be read out after the sterilization process.

In step 150, the sterilized unit is collected (inventorized) and stored, before in step 160 it is shipped to a customer in due course.

The one-time use of the single use bioreactor system at the customer is represented by step 170.

The outlined life cycle by no means is to be understood in a limiting sense and of course can include further steps.

Before the step 130 of packaging, the integrity of the single use bioreactor system can be checked on the part of the manufacturer, as will yet be explained in detail below. In addition, the product-relevant data can be read out from the memory of the RFID tag at any time by means of a suitable reading device and be evaluated in connection with an inventory or quality check or the like. Finally, data can be changed and/or additional data can wirelessly be written into the memory of the RFID tag when necessary.

In the following, the performance, documentation and storage of the test or test results of an integrity check of the single use bioreactor system will be described more exactly. For checking the integrity of the single use bioreactor system the following aspects (individually or in any combination) can be used in principle: physical integrity (tightness) of the container (bag); material validation incl. qualification parameters (extractables and leachables, determination of the total bacteria count (bioburden) etc.; compliance with the environmental conditions for transport and storage (e.g. limit temperature) including date of manufacture and use-by date; traceability from the production via the warehouse to the site of use; matching as to whether components of the single use bioreactor system fit together (bag and hardware such as holder, etc.); parameters in the documentation, e.g. batch release. The corresponding information on the integrity of the entire system is stored on the memory of the RFID tag, which can be integrated into the container and be operated and read out wirelessly.

In the following, an integrity check and documentation will be described by way of example with reference to a container (bag) for use in a bioreactor. For carrying out the tests a suitable testing device is used, such as the above-mentioned “Sartocheck 4 plus Bag Tester” by means of which a pressure test can be carried out and a cGMP-compliant documentation can be created. The testing device is equipped with a transmitting/receiving unit for RFID tags, which is integrated into the entire system control unit of the testing device or the testing process.

The tests provided for the integrity check are carried out in the usual way by means of the testing device. The test results, including the time of the performance and the framework conditions of the integrity check, however are not only stored in a data base as customary so far (in general together with earlier and future test results of other components tested already or yet to be tested). The test results in addition are written into the memory of the RFID tag integrated into the container. Thus, the product-relevant data and the test results remain on a single use component of the bioreactor system, here on the container, and can be read out and completed when necessary.

For a traceability of the single use bioreactor system in the production and/or storage and/or with regard to the monitoring of environmental influences and/or the compliance with specified storage conditions it is helpful when the RFID tag is permanently connected to an energy storage device.

The obvious measure in this connection, i.e. to integrate an energy storage device into the RFID tag itself, is problematic because of the gamma radiation provided for the sterilization. It is known to the skilled person that usual batteries and accumulators, such as e.g. lithium-ion accumulators, are damaged or impaired by the gamma irradiation to such an extent that their capacitance decreases significantly.

In principle, it is possible to design the energy storage device (battery, accumulator) so large that even a drastic reduction of the capacitance still is sufficient for the intended purposes. Another possibility for a permanent power supply is the use of a (largely) gamma-radiation-resistant energy storage device or the targeted compliance with a specified radiation dose window by means of a dosimeter. The specifications and the actual parameters of the irradiation can be stored in the memory of the RFID tag.

In addition, the single use bioreactor system can be completed by a rechargeable electric energy storage device (accumulator). An example of this kind of power supply is shown in FIGS. 2 and 3. An RFID tag 200, which optionally can be equipped with its own battery 210, is integrated into a container (bag) 220 for a bioreactor. The container 220 is disposed in a flexible overwrap (not shown here). An accumulator unit 260 electroconductively connected to the RFID tag 200 via a cable 240 and a plug connection 250 or the like in a pocket is attached to the inner side of a rigid carton 280 in which the container 220 is packed with its overwrap. The accumulator unit 260 is provided with a device for wireless charging 290, such as a suitable near field communication (NFC) antenna or another suitable receiving or coupling unit, preferably according to an established standard. Upon completion or at a later date before the radiation sterilization, the accumulator unit 260 for the first time is charged by a suitable external energy source in a contactless way.

The use of the accumulator unit 260 allows to document relevant events and ambient conditions in the history of the single use component, here of the container 220, or of the entire single use bioreactor system packed in the carton 280 in the memory of the RFID tag 200 not only on a one-time basis, but over time, in particular in the time before a delivery to a customer. The collection and storage of the data can be effected in regular or irregular intervals. The data can be read out from the memory of the RFID tag 200 in a contactless way at any time by means of a suitable reading device.

For the collection of the events and/or ambient conditions single use probes possibly can be used, which anyway are part of the packed bioreactor system and are provided with one or more suitable sensors, such as a temperature sensor, a humidity sensor and/or a radiation sensor.

From the stored data conclusions as to the current state can be made later on and statements can be made as to whether and possibly how long the components of the single use bioreactor system still are suitable for the intended use.

As mentioned already, the accumulator unit 260 can suffer a partial damage due to the radiation sterilization, which leads to a complete or partial discharge. If the energy of the accumulator unit 260 no longer is sufficient for the intended tasks, this point in time is documented, i.e. written into the memory of the RFID tag, and the internal battery 210 of the RFID tag, if present, takes over the further power supply.

Owing to the device 290 for wireless charging it is possible in principle, if necessary, to again charge the accumulator unit 260 a contactless way from outside by an external energy source after the radiation sterilization, as is shown in FIG. 1 by step 180. Possibly, the frequency of storing data and reading out stored data can also be decreased or adapted in order to ensure the coverage of a desired minimum period of time.

The electrically conductive connection between the RFID tag 200 and the accumulator unit 260, here the cable 240, ideally is designed such that upon opening of the carton 280 or the overwrap or upon removal of the content of the overwrap or in the case of a defined concussion, the electrically conductive connection is interrupted. If present, the internal battery 210 of the RFID tag 200 then takes over the further energy supply. The time of the interruption is documented, i.e. written into the memory of the RFID tag 200, and can be interpreted as an indication that the single use bioreactor system is unpacked and utilized or that the integrity of the single use bioreactor system no longer is given.

Before shipment to the customer a write protection of the memory can be activated in the RFID tag 200 in order to protect from manipulations or an inadvertent deletion of data that are stored in the memory. It thereby is ensured that the correct data are permanently available. The customer himself can read out the data when necessary and correspondingly use the same for his purposes. Another memory area can, however, be provided to the customer as a (re-)writable memory so that the customer can store his own data in the RFID tag 200 and read them out.

1. A single use bioreactor system, sterilizable by irradiation, for use in a biopharmaceutical process, the single use bioreactor system comprising an RFID tag and a memory rewritable in principle, in which data with respect to an integrity check of the single use bioreactor system are stored.

2. The single use bioreactor system according to claim 1, characterized in that the memory is part of the RFID tag.

3. The single use bioreactor system according to claim 1, characterized in that the memory comprises a ferroelectric random access memory chip.

4. The single use bioreactor system according to claim 1, characterized in that product-relevant data are stored in the memory.

5. The single use bioreactor system according to claim 4, characterized by a write protection which after activation prevents deletion and overwriting of the data stored in the memory.

6. The single use bioreactor system according to claim 1, characterized in that the memory includes a free, writable area and a blocked area that no longer is writable.

7. The single use bioreactor system according to claim 1, characterized in that the RFID tag is provided with an internal battery.

8. The single use bioreactor system according to claim 1, comprising one or more sensors for detecting certain events and/or ambient conditions.

9. An apparatus assembly comprising a single use bioreactor system according to claim 1 and an accumulator unit attached to a packaging outside the single use bioreactor system, which in a releasable way is electroconductively connected to the RFID tag.

10. The apparatus assembly according to claim 9, characterized in that the accumulator unit includes a device for wireless charging by an external energy source.

11. The apparatus assembly according to claim 9, characterized in that the accumulator unit is attached to an inner side of an outer packaging.

12. The apparatus assembly according to any of claim 9, comprising a writing device that is configured for the wireless writing of data into the memory of the single use bioreactor system, and a reading device that is configured for wireless reading out of data stored in the memory.

13. A method for quality assurance of a single use bioreactor system that is provided for use in a biopharmaceutical process, the method comprising the following steps:
providing the single use bioreactor system with an RFID tag and a memory rewritable in principle;
carrying out an integrity check of at least one component of the single use bioreactor system before sterilization of the single use bioreactor system by irradiation; and
writing data with respect to the integrity check into the memory before sterilization of the single use bioreactor system.

14. The method according claim 13, characterized in that before shipment of the single use bioreactor system to a customer a write protection of the memory is activated.

15. The method according claim 13, characterized in that the single use bioreactor system detects events and/or ambient conditions determined by means of one or more sensors and writes associated data into the memory, wherein the single use bioreactor system is supplied with energy via an external accumulator unit.

16. The method according to claim 15, characterized in that an internal battery of the RFID tag takes over the energy supply as soon as the energy of the accumulator unit falls below a limit value.

17. The method according to claim 15, characterized in that after radiation sterilization the accumulator unit is charged again in a contactless way from outside by an external energy source.

18. The method according to any of claim 15, characterized in that upon opening of a packaging to which the accumulator unit is attached, upon removal of the single use bioreactor system from the packaging or in the case of a defined concussion the electric line between the accumulator unit and the single use bioreactor system is interrupted and the time of this event is written into the memory.

19. The single use bioreactor system according to claim 4, characterized in that the product-relevant data stored in the memory comprises one or more of a product identification code, a date of manufacture or a use-by date.

20. The method according to claim 13 wherein the RFID tag and a memory rewritable in principle comprises a ferroelectric random access memory chip, which is part of the RFID tag.
